(19) United States
(12) Patent Application Publication
(10) Pub. No.: US 2008/0050827 A1
(43) Pub. Date: Feb. 28, 2008

(54) Derivatization-enhanced analysis of amino acids and peptides

(76) Inventors: Hamid Mirzaei, Seattle, WA (US); Fred E. Regnier, West Lafayette, IN (US); Wen-Chu Yang, West Lafayette, IN (US)

Correspondence Address: BRINKS HOFER GILSON & LIONE, P.O. BOX 10395, CHICAGO, IL, 60610, US

(21) Appl. No.: 11/824,698

(22) Filed: Jul. 2, 2007

Related U.S. Application Data
(63) Continuation of application No. PCT/US07/08692, filed on Apr. 6, 2007.
(60) Provisional application No. 60/789,641, filed on Apr. 6, 2006.

(51) Int. Cl. G01N 33/68; G01N 33/00; G01N 37/00

(52) U.S. Cl. 436/8; 436/56; 436/86; 436/89

(57) ABSTRACT

The present invention provides compositions and methods for enhanced detection and quantification of amino acids by derivatization. Also provided are compositions and methods for enhanced detection and quantification of peptides by derivatization.

### CROSS-REFERENCE TO RELATED APPLICATIONS

The present application is a continuation of PCT Patent Application Serial No. PCT/US2007/008692, filed Apr. 6, 2007, which claims priority to U.S. Provisional Application Ser. No. 60/789,641, filed Apr. 6, 2006, both of which are incorporated herein by reference.

### FEDERALLY SPONSORED RESEARCH OR DEVELOPMENT

This invention was made with United States government support from the National Institutes of Health (NIH), grant number AG13319. The United States government may have certain rights in this invention.

### TECHNICAL FIELD

The present invention relates to the field of methods for the quantitative determination of amino acids and peptides.

### BACKGROUND

The evolution of methods for amino acid analysis has closely paralleled the development of analytical tools for the life sciences during the past 50 years. First there were the paper chromatography methods, then the ion exchange method with post-column ninhydrin-based detection that led to HPLC, and finally reversed phase chromatographic (RPC) separation methods with pre-column derivatization. Derivatization of amino acids with reagents such as o-phthalaladehyde (OPA)-mercaptoethanol, dansyl chloride (Dns-CI), phenyl isothiocyanate (PITC) and its analogs, fluorenylmethyl chlorofomate (FMOC), 6-aminoquiline carbamate (6-AQC), vastly increased detection sensitivity.

Mass spectral methods are currently playing a role in the detection of amino acids. Mass spectrometry (MS) has numerous advantages: analytes do not have to be chromatographically resolved to allow detection, and multiple dimensions of structure analysis are available through MS/MS analysis without adding time to the analysis. Moreover, extracted ion chromatograms allow multiple mass-related features of a mixture to be recognized, quantified, and displayed. This technology enables the detection of both parent ions and fragment ions common to multiple species.

However, there are problems with the reversed phase chromatography-mass spectrometry (RPC-MS) approach. One problem is that matrix effects can suppress the ionization of amino acids. This is particularly true of amino acids that do not ionize well in the first place. Yet another problem is that polar amino acids elute from RPC columns unretained in the column void volume with a large number of other substances. This problem has been addressed by derivatization with a more hydrophobic group such as benzoic acid or 2,4-dinitrofluorobennzene (DNFB), to increase RPC retention of amino acids. Unfortunately, derivatization can also impact ionization efficiency. For example, although all nineteen DNFB derivatized amino acids examined were well resolved by RPC, only H, R, and K, were observed in the positive ion mode of electrospray ionization-mass spectrometry (ESI-MS) approach (Liu et al., 2004, Rapid Commun. Mass Spectrum. 18: 1059-1065). This is apparently due to a loss in charge caused by derivatization.

A strategy for dealing with poor ionization of amino acids is to derivatize them with a group that is easily protonated, such as a tertiary amine. For example, dansyl derivatives are more easily protonated during electrospray ionization (ESI) because of the dimethylamino moiety in the naphthyl ring. Derivatizing amino acids with dimethylformamide dimethylacetal (DMF-DMA) also enhances detection in tandem mass spectrometry, especially for smaller amino acids such as glycine. Derivatization with N,N-dimethyl-2,4-dinitro-5-fluorobenzylamine (DMDNFB) is another example in which a robust mass signal is obtained by introduction of a tertiary amine. Quaternization could increase ionization efficiency even more as has been shown with steroids and peptides.

Protein identification in proteomics is achieved in several ways. One is to tryptically digest a proteome and, after several dimensions of chromatographic fractionation, identify peptide cleavage fragments by electrospray ionization-mass spectrometry (ESI-MS). However, because a tryptic digest of even a simple proteome can contain several hundred thousand peptides, chromatographic fractions being introduced into an ESI-MS can contain hundreds to thousands of components. This causes a problem in ESI-MS. As the complexity of samples being introduced into the instrument increases, many peptides fail to ionize because of a phenomenon known as matrix suppression of ionization.

Passage of peptides from droplets formed during the electrospray process into the gas phase is the result of peptide desolvation. Peptide hydrophobicity seems to play a role in desolvation, probably because hydrophobicity dictates the rate at which peptides migrate to the surface of droplets. To be detected, these gas phase peptide species need to acquire charge. Obviously basic peptides are more likely to acquire a proton and ionize. Attaching a quaternary amine to peptides enhances ionization by providing a permanent positive charge. When hydrophobicity and good gas-phase proton affinity are combined in hydrophobic, cationic peptides, these peptides ionize more readily along with suppression of the ionization of other peptides. Addition of tetramethylammonium bromide to a solution of peptides being electrosprayed suppresses the ionization of all peptides (Pan and McLuckey, 2003, Analytical Chemistry 75: 5468-5474). This means that surfactant-like species can saturate the droplet surface and push peptides toward the interior of droplets. It also explains how hydrophobic peptides derived from more abundant proteins suppress ionization. They diminish or eliminate droplet surface area for other peptides to be protonated and pass into the gas phase.

It would be advantageous to provide new methods for enhanced analysis of amino acids and peptides, to increase ionization efficiency, or to enhance quantification of amino acids and peptides in metabolomics. The invention described here addresses these and related needs.

### SUMMARY OF THE INVENTION

The present invention provides a reagent comprising an N-alkyl-nicotinic acid N-hydroxysuccinimide ester (Cₙ-NA-NHS). The ester may include deuterium atoms.

The present invention also provides a method for analyzing an amino acid. The method includes contacting the amino acid with an N-alkyl-nicotinic acid N-hydroxysuccinimide ester (Cₙ-NA-NHS) and detecting the resultant derivative a light absorption method.

The present invention provides a method for affixing a tag to amino acid. The method includes contacting the amino acid with a derivatization reagent comprising an N-hydroxysuccinimide ester of N-alkyl-nicotinic acid (Cₙ-NA-NHS).

The present invention provides a reagent that includes a quaternary amine, an n-octyl chain bonded to the quaternary amine, and a peptide binding group. The reagent covalently binds to a peptide through the peptide binding group.

The present invention provides a method for affixing a tag to a peptide. The method includes contacting the peptide with a reagent that includes a quaternary amine with an n-octyl chain bonded to the quaternary amine, the reagent having a peptide binding group, the reagent covalently binding to the peptide through the peptide binding group.

The present invention also provides a method for analyzing a peptide. The method includes contacting the peptide with a reagent to derivatize the peptides, and detecting the resultant derivatives by a light absorption method. The reagent comprising a quaternary amine, an n-octyl chain bonded to the quaternary amine, and a peptide binding group. The reagent covalently binds to a peptide through the peptide binding group.

### BRIEF DESCRIPTION OF THE DRAWINGS

FIG. 1 is a scheme illustrating the synthesis of N-alkyl-nicotinic acid N-hydroxysuccinimide ester (Cₙ-NA-NHS).

FIG. 2 is a scheme illustrating the reactions involved in the derivatization of amino acids with Cₙ-NA-NHS and subsequent hydrolysis of the unused derivatizing agent.

FIG. 3 is a graph showing the effects of alkyl chain length on retention time of derivatized amino acids.

FIG. 4 is a graph showing a reverse phase chromatogram from 18 amino acids derivatized with C₄-NA-NHS using 1-minute incubation time.

FIG. 5 is a graph showing the extracted total ion chromatogram from 18 amino acids derivatized with C₄-NA-NHS using 10-minute incubation time.

FIG. 6 is a graph showing the mass spectra of NA- and C₁₋₄-NA derivatives of tryptophan.

FIG. 7 is a graph showing the mass spectra of C₄H₉-NA-Phe and C₄D₉-NA-Phe at a 1:1 molar ratio.

FIG. 8 is a graph showing the chromatographic isotope effects for three amino acids derivatized with C₄H₉-NA-NSH and C₄D₉-NA-NSH at the ratio of 1:1.

FIG. 9 illustrates the structures of the QAT and C₈-QAT reagents.

FIG. 10 is a graph depicting reversed-phase chromatograms for 1) unlabeled and 2) C₈-QAT labeled transferrin digest separated on a C₁₈ column.

FIG. 11 is a graph showing ion chromatograms for: 1) QAT-labeled and 2) C₈-QAT labeled transferrin digests separated on a C₈ reversed-phase column.

FIG. 12 is a graph showing the extracted ion chromatograms for native, QAT and C₈-QAT labeled model peptide Ac-Gln-Lys-Arg-Pro-Ser-Gln-Arg-Ser-Lys-Tyr-Leu-OH.

FIG. 13 is a graph depicting the MS/MS spectrum for the peptide MYLGYEYVTAIR.

### DETAILED DESCRIPTION OF THE PREFERRED EMBODIMENTS

Unless defined otherwise, all technical and scientific terms used herein have the meaning commonly understood by a person skilled in the art to which this invention belongs.

The present invention provides methods for the analysis of amino acids. The methods include derivatization of amino acids by N-acylation with an acid that is also hydrophobic and has a quaternary amine. Generally the amino acid standards and samples are derivatized with an N-alkyl-nicotinic acid N-hydroxysuccinimide ester, to yield a stable amino acid derivative, which is separated by reversed phase chromatography. Such derivatization can facilitate RPC/MS analysis of amino acids in several ways. One way in which the analysis of amino acid is facilitated is by increasing the retention of small hydrophilic amino acids during reversed phase chromatography sufficiently to cause them to elute beyond the void volume peak. This is preferred to resolve them from large amounts of other interfering polar, non-retained substances in complex samples. A second way in which the analysis of amino acid is facilitated is to aid in electrospray ionization by increasing the charge on amino acids through the introduction of a quaternary amine group. A third way in which the analysis of amino acid is facilitated is by combining the effects of having both a hydrophobic and quaternary amine groups in close proximity. Lengthening the alkyl chain in the hydrophobic quaternary amine portion of derivatized amino acids increases their surface active properties and directs them to the surface of electrospray droplets where ionization is more likely to occur.

The present invention provides for the introduction of stable isotope coding of amino acids according to sample origin during the course of in vitro derivatization. This step increases the ionization efficiency of amino acids. This is in effect a type of molecular bar coding that allows a unique mass code to be placed on samples from different sources. It also greatly facilitates comparative quantification studies. In one example, subsequent to the mixing of differentially coded samples, it is possible to determine the relative concentration of individual amino acids between these samples in a single analysis. Thus, the methods of the present invention provide for relative quantification that should be of value in comparative metabolomics.

The present invention provides new compounds useful for analysis of amino acids and peptides. In one example, the novel compounds are N-alkyl-nicotinic acid N-hydroxysuccinimide esters (Cₙ-NA-NHS), which are used as amino acid derivatizing agents as described herein. In another example, the novel compounds are quaternary amines with adjacent n-octyl chains, which are used as peptide derivatizing agents as described herein.

“Analysis” of amino acids includes, but is not limited to, detection, identification, quantitative and qualitative determination, separation into component parts, and other examination of amino acids. “Analysis” of peptides includes, but is not limited to, detection, identification, quantitative and qualitative determination, separation into component parts, and other examination of peptides.

The present invention provides new methods for amino acid analysis, involving derivatization of amino acids with an N-hydroxysuccinimide ester of N-alkyl-nicotinic acid (Cₙ-NA-NHS). The derivatization may be followed by reversed phase chromatography and electrospray ionization mass spectrometry (RPC-MS). The derivatized amino acids are detected using detection methods known in the art, including light absorbance, e.g., UV light or fluorescence-based detection methods.

“Derivatization” refers to the conversion of a chemical compound into a derivative. This is often done for purposes of identification of the chemical compound. Thus, derivatization of an amino acid refers to the conversion of the amino acids into a derivative. Derivatization of a peptide refers to the conversion of the peptide into a derivative. Derivatizing “agent” (also referred to as derivatizing “reagent”) refers to a compound used because of its chemical or biological activity with respect to derivatization.

In one example of the present invention, the detection sensitivity of amino acids increases as the N-alkyl chain length of the nicotinic acid derivatizing agent is increased from 1 to 4. In one example, N-acylation of amino acids with the Cₙ-NA-NHS reagents in water produces stable products in about one minute. This can be achieved, for example, using a 4-fold molar excess of derivatizing agent in 0.1 M sodium borate buffer at pH values ranging from 8.5 to 10. Using this method, some O-acylation of tyrosine can also be observed but the product hydrolyzes within a few minutes at pH 10. The cystine product also degrades slowly over the course of a few days due to reduction of the disulfide bond to form cysteine.

The retention time of Cₙ-NA derivatized amino acids can be lengthened in reversed phase chromatography (RPC) to the extent that polar amino acids are retained beyond the solvent peak, particularly in the cases of the C₃-NA and C₄-NA derivatives. Complete resolution of 18 amino acids can be achieved in as little as 28 minutes using the C₄-NA-NHS reagent of the present invention. Compared to N-acylation with benzoic acid, derivatization with C₄-NA-NHS increased MS detection sensitivity by 6-fold to 80-fold. This can probably be attributed to the surfactant properties of the Cₙ-NA-NHS reagents. The quaternary amine increases the charge on amino acid conjugates while the presence of an adjacent alkyl chain further increases ionization efficiency by enhancing amino acid migration to the surface of electrospray droplets.

The present invention provides for modification of Cₙ-NA-NHS reagents with deuterium in order to prepare coded sets of derivatizing agents. These coding agents can be used to differentially code samples and, after mixing, carry out comparative concentration measurements between samples using extracted ion chromatograms, to estimate relative peak areas of derivatized amino acids.

The present invention further provides methods for the analysis of peptides. In one example, the addition of a tag containing a quaternary amine with an adjacent n-octyl chain to primary groups in peptides can increase the electrospray ionization efficiency of many peptides by 10-fold or more. This enhancement of ionization efficiency occurs most consistently with peptides under about 500 Daltons (Da), but can have an equally large impact on larger peptides as well. The most dramatic increases in ionization efficiency following derivatization are likely to occur with peptides that are difficult to ionize in the native state. Smaller degrees of augmentation are likely in peptides that ionize well in the un-tagged form. Not wanting to be bound by the following explanation, improvements in ionization efficiency following C₈-QAT tagging are most likely due to increased accumulation of peptides at droplet surfaces by imparting surfactant properties to peptides.

Derivatization of peptides with reagents like C₈-QAT can improve detection sensitivity of many peptides while minimizing differences in ionization efficiency. This may be particularly useful in the analysis of complex peptide mixtures such as those derived from tryptic digests of a proteome.

The present invention provides methods for affixing a “tag” (also referred to as a “label”) to an amino acid or a peptide. As used herein, “tag” or “label” refers to a derivatization reagent used for identification of an amino acid. For example, affixing a tag to an amino acid includes contacting the amino acid with a derivatization reagent comprising an N-hydroxysuccinimide ester of N-alkyl-nicotinic acid (Cₙ-NA-NHS), the reagent having an amino acid binding group, to covalently bind the reagent to the amino acid and thereby form a derivatized amino acid. Affixing a tag to a peptide includes contacting the peptide with a derivatization reagent comprising a quaternary amine with an n-octyl chain bonded to the quaternary amine, the reagent having a peptide binding group, the reagent covalently bind to the peptide through the peptide binding group.

Electrospray ionization of peptides is governed by two major factors. One is the ability of the peptide to acquire charge (generally a proton) before or during escape from the surface of electrospray droplets. The other factor is the peptides' surface activity, i.e., the ease with which the peptide migrates to the droplet surface. The rationale in the design of the C₈-QAT labeling agent was that introduction of a permanent positive charge into peptides and addition of an adjacent aliphatic hydrocarbon tail would augment both of these processes. In most cases quaternization does in fact improve ionization efficiency. It is also shown that adding an octyl side chain to the quaternary amine can have a very large impact on ionization efficiency.

Ideally it would be possible to look at the structures of a family of peptides and predict their relative ionization efficiency before and after derivatization with a reagent such as the C₈-QAT. Generally the ionization efficiency of peptides that ionize well in their native form is not improved substantially by derivatization. In contrast, using the methods of the present invention, ionization of many peptides that ionized poorly in their native form can be improved an order of magnitude or more through derivatization with the C₈-QAT reagent.

The present invention provides for improvement of the ionization efficiency of peptides smaller than about 500 Daltons (Da) by an order of magnitude or more with derivatization. A relatively minor change in peptide concentration can change the relative ionization efficiency of peptides in a mixture. Tagging peptides with C₈-QAT moderates this effect.

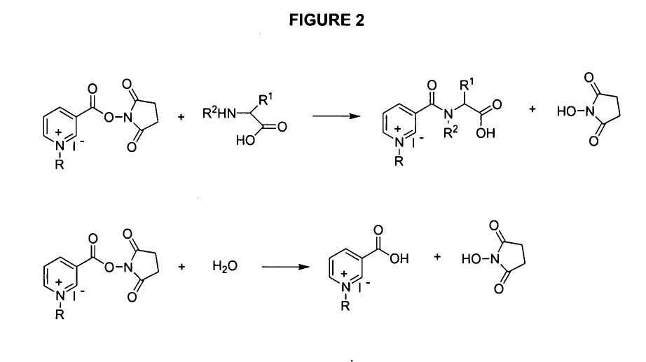

The present invention provides for amino acid derivatization by amide bond formation through nucleophilic displacement of N-hydroxysuccinimide (NHS) from an N-alkylnicotinic acid (NA) by the α-amino group of the amino acid. The reaction scheme is shown in FIG. 2. Amino acid derivatization occurs rapidly. Hydrolysis of Cₙ-NA-NHS in contrast is a much slower reaction.

In the case of lysine, the ε-amino group is sufficiently nucleophilic at slightly basic pH such that it is also derivatized. Beyond being easily displaced, an advantage of NHS activation is that derivatization can be achieved in water. This is a great benefit because amino acids are water soluble.

A similar protocol for α-amino group derivatization is used with peptides where it has been shown that NHS-activated nicotinic acid is sufficiently stable in water to quantitatively acylate primary amine groups before finally hydrolyzing (Munchbach et al., 2000, Anal. Chem. 72: 4047-4057). The reagent used for derivatization of amino acids differs from the one used in peptide derivatization in the addition of alkyl groups (Cₙ) to nicotinic acid (NA) and quaternization of the ring nitrogen.

N-Alkylnicotinic acid reagents activated with NHS are herein represented by the general formula Cₙ-NA-NHS. As used herein, “activation” refers to making the molecules reactive or more reactive. For example, activation of an N-alkylnicotinic acid reagent with NHS refers to making the N-alkylnicotinic acid reagent more reactive for purposes of derivatization of amino acids. Also for example, placing deuterium atoms in the reagents of this invention used for derivatization of peptides may activate these reagents for purposes of derivatization of peptides.

Hydrolysis of residual Cₙ-NA-NHS after derivatization can be accelerated by adjusting pH to about 10 with hydroxylamine (Wang and Regnier, 2001, J. Chromatogr. A. 924: 345-357). Hydrolysis is achieved in less than one minute under these conditions to yield Cₙ-NA according to MS analysis. Cₙ-NA-NHS derivatizing agents can be stored in acetonitrile to preclude premature hydrolysis.

It is to be understood that this invention is not limited to the particular methodology, protocols, subjects, or reagents described herein, and as such may vary. It is also to be understood that the terminology used herein is for the purpose of describing particular embodiments only, and is not intended to limit the scope of the present invention, which is limited only by the claims. The following examples are offered to illustrate, but not to limit, the claimed invention.

### EXAMPLES

Materials. An amino acid standard mixture was obtained from Sigma-Aldrich (St. Louis, Mo.). The following amino acids including ammonium chloride were in the mixture: L-alanine, L-arginine, L-aspartic acid, L-cystine, L-glutamic acid, glycine, L-histidine, L-isoleucine, L-leucine, L-lysine, L-methionine, L-phenylalanine, L-proline, L-serine, L-threonine, L-tyrosine, and L-valine. Amino acids in this standard mixture were at a concentration of 2.5 μmoles/ml in 0.1 N HCl. The exception was L-cystine (at 1.25 μmoles/ml). Anhydrous acetonitrile (ACN), dicyclohexylcarbodiimide (DCC), alkyl iodide, N-hydroxysuccinimide, and dimethylformamide (DMF) were also purchased from Sigma-Aldrich (St. Louis, Mo.). HPLC grade acetonitrile (ACN), urea, and acetone were obtained from Mallinkrodt Baker (Phillipsburg, N.J.). Sequanal grade trifluoroacetic acid (TFA) was obtained from Pierce (Rockford, Ill.). 1-Iodobutane-d9 was the product of Cambridge Isotope laboratories (Andover, Mass.). Double-deionized water was produced by a Milli-Q gradient A10 system Millipore (Bedford, Mass.).

Synthetic peptides Ac-Gln-Lys-Arg-Pro-Ser-Gln-Arg-Ser-Lys-Tyr-Leu-OH, H-Asp-Arg-Val-Tyr-Ile-His-Pro-Phe-His-Leu-OH, H-Ala-Phe-Pro-Leu-Glu-Phe-OH, H-Ser-Tyr-Ser-Met-Glu-His-Phe-Arg-Trp-Gly-OH, H-Cys-Asp-Pro-Gly-Tyr-Ile-Gly-Ser-Arg-OH, H-Tyr-Gly-Gly-Phe-Met-Lys-OH, H-Pro-His-Pro-Phe-His-Phe-His-Phe-Phe-Val-Tyr-Lys-OH, H-Ala-Leu-Gly-OH, H-Ala-Gly-Gly-OH, H-Ala-Met-OH, and H-Ala-Ser-OH were purchased from BACHEM Bioscience Inc. (King of Prussia, Pa.). Dithiothreitol (DTT), trypsin, N-alpha-tosyl-L-lysine chloromethyl ketone (TLCK), apo-transferrin (human), 4-iodobutyric acid, N-hydroxysuccinimide (NHS), dicyclohexylcarbodiimide (DCC), 4-aminobutanoic acid, potassium bicarbonate, methyl iodide, chloroform and N,N-dimethyl-N-octylamine were obtained from Sigma-Aldrich Chemical Co. (St. Louis, Mo., USA).

218TP54 reversed-phase C₁₈ column was purchased from Vydac (W. R. Grace & Co.-Conn., Columbia, Md.). DE44H10426 Zorbax reversed-phase C₁₈ column (0.5×150 mm) and DE45C00085 Zorbax reversed-phase C₈ column (0.3×150 mm) were purchased from Agilent Technologies, Inc. (Palo Alto, Calif.). Reversed-phase chromatography (RPC) analyses were done on a BioCAD 20 Micro-analytical Workstation (PE Biosystems, Framingham, Mass.). The LC system used in conjunction with mass spectrometer was an Agilent 1100 series instrument. LC/MS mass spectral analyses were done using a Sciex QSTAR hybrid LC/MS/MS Quadrupole TOF mass spectrometer (Applied Biosystems, Foster City, Calif.). All spectra were obtained in the positive ion mode.

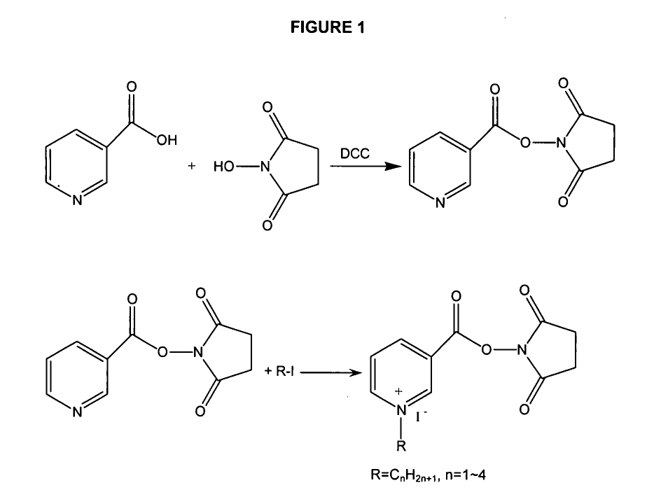

Synthesis of N-hydroxysuccinimide activated N-alkyl-nicotinic acid esters (Cₙ-NA-NHS). The scheme for synthesizing N-alkyl-nicotinic acid N-hydroxysuccinimide ester (Cₙ-NA-NHS) derivatizing agents is shown in FIG. 1. The procedure is an adaptation of a method first described by Tedjamulia and Srivastava, 1985, J. Med. Chem. 28: 1574-1580. An aliquot of dicyclohexyl carbodiimide (DCC) (44 mmol, 9.0 g) was added to 5 ml of dry DMF containing 40 mmol (5.0 g) of nicotinic acid and 50 mmol of N-hydroxysuccinimide (4.6 g). The reaction mixture was stirred at room temperature for 48 hours and then treated with glacial acetic acid (0.2 ml) to decompose excess DCC. After allowing the mixture to stir at room temperature for an additional hour, the temperature was reduced to 3-4° C. and stirring continued for another 4 hours.

Precipitated dicyclohexylurea was removed by filtration and the filtrate evaporated under vacuum to reduce the volume to about 10 ml. A pale yellow product was precipitated by addition of a mixture of hexane and isopropanol (6:1 in volume). The nicotinic acid N-hydroxysuccinimide ester precipitate was dried under vacuum and used to prepare a series of N-alkyl-nicotinic acid N-hydroxysuccinimide esters (alkyl=C₁, C₂, C₃, C₄) by direct quaternization. Five mmol nicotinic acid N-hydroxysuccinimide ester were added to 10 mmol of an alkyl iodide in 5 ml of dry acetonitrile (ACN). The mixture was refluxed at about 70-75° C. under argon for about 24 to 48 hours. A yellow product precipitated from the solution either during the reaction or after removing some of the solvent, depending on the alkyl groups. After cooling, the Cₙ-NA-NHS product was collected by filtration, washed with cold acetone, and dried to yield yellow crystals that were stored in a desiccator until used for amino acid derivatization.

Derivatization of Amino Acids. A Cₙ-NA-NHS solution was prepared at a concentration of 40.0 mg/ml in dry ACN. This solution is stable for 5 days at ambient temperature or at least one month at 4° C. Two types of standard solutions were used in optimization studies. One type contained 50.0 μmol/ml of an individual amino acid in 0.1 mol/L hydrochloride. Tryptophan was an exception in that it was dissolved in deionized water instead of HCl. The other type of standard solution was made by adding 10.0 μl of all the individual amino acid solutions in HCl and 0.5 μL of 50.0 μmol/ml tryptophan in deionized water to 200 μL of 0.1 mol/L sodium borate buffer at pH 9.0. After mixing, 20 μl of 40.0 mg/ml Cₙ-NA-NHS (roughly a 4-fold molar excess) was added. The reaction was allowed to proceed by one minute before RPC analysis. Samples of unknown amino acid concentration were analyzed by adding 10.0 μl of sample and 20 μl of 40.0 mg/ml Cₙ-NA-NHS solution to 200 μL of 0.1 mol/L sodium borate buffer at pH 9.0. After a minimum of one minute incubation derivatized amino acids were analyzed by RPC-MS. Incubation times of 10 minutes were used when the objective was to allow O-acylated tyrosine to hydrolyze to a single N-acylated product.

Amino acid (AA) derivatization was achieved by amide bond formation through nucleophilic displacement of N-hydroxysuccinimide (NHS) from an N-alkylnicotinic acid (NA) by the α-amino group of the AA, according to the reaction scheme shown in FIG. 2.

Optimization of derivatization was examined using the C₃-NA-NHS reagent and tryptophan (Trp) with RPC-MS analysis of the reaction products. Peaks eluting at 7.5, 13.1, 16.8 minutes were found to be of m/z 166.1(+1), 204.2(+1), and 352.1(+1), respectively. The number in parentheses behind the m/z value is the charge state of the ion observed in the MS spectrum. These three peaks were identified as C₃-NA, free Trp and C₃-NA-Trp, according to their retention times and m/z values. The derivatization reaction was investigated by carrying the reaction out in 200 μL of 0.1 mol/L sodium borate buffer at pH 8.0 to which 10 μl of a 50 μmol/ml tryptophan solution was added along with 20 μl of the derivatization reagent. C₃-NA-NHS and Trp were mixed in 1:1 and 3:1 molar ratios and the reaction was allowed to proceed for 5 minutes. Although some Trp was seen at the 1:1 C₃-NA-NHS/Trp ratio, at the 3:1 molar ratio Trp derivatization was complete in 30 seconds. A 1 minute reaction time and 4:1 molar ratio were used in subsequent experiments to assure that derivatization would be complete.

Further optimization of the reaction was carried out with a standard 17 amino acid mixture from Sigma using the C₃-NA-NHS derivatizing agent. Yields were studied using 0.1 M sodium borate buffers ranging in pH from 7.5 to 10. Although the yield increased slightly between pH 7.5 and 8.5, there was little additional increase in yield above pH 8.5. Similar findings have been reported with other acylating agents.

In addition to N-acylation, Tyr showed O-acylation in the pH range from 7.5 to 8.5. Three peaks containing Tyr were seen in the RPC tracing. One of the Tyr products was the expected α-amino group acylation product. Another was from acylation of the phenolic —OH. O-Acylation of tyrosine has also been reported in the case of peptides where O-acylated products were hydrolyzed after derivatization by the addition of hydroxylamine. The third arose from derivatizing both the phenolic hydroxyl and the α-amino group.

At pH 7.5 the phenolic hydroxyl derivative was dominant with about 95% of the derivatization occurring at this site. At pH 8.0 greater than 90% of the Try product resulted from dual derivatization. When the reaction was carried out at pH 9.0, the α-amino derivatized product dominated the mixture. Thus, derivatization of the phenolic hydroxyl group occurred faster than that of the a-amino group, but was also being hydrolyzed. Given enough time at the higher pH, the main product at pH 9.0 should be from derivatization on the α-amino group. Indeed, after 10 minutes incubation at pH 9.0, the main product was from a-amino group derivatization. Neither buffer concentration nor reagent to amino acid ratio had much influence on product distribution and yield under these conditions. As a consequence of the addition of Tyr, derivatization reactions were carried out in 0.1 M sodium borate at pH 9.0 for 10 minutes at a reactant to analyte ratio of no less than 4:1. Although higher pH also favors hydrolysis of NHS esters, the rate of derivatization exceeded the rate of hydrolysis in the early phase of the reaction. Table 1 shows the m/z value, retention time and reproducibility of derivatization (% RSD of peak area) for C₄-NA derivatives of amino acids.

TABLE 1
The m/z value, retention time and reproducibility of derivatization
(% RSD of peak area) for C₄-NA derivatives of amino acids

| | | Retention time | Peak-area RSD |
|---|---|---|---|
| Amino acid | m/z | (min) | (%, n = 10)^{a} |
| His | 317.02 (+1) | 11.89 | N.D^{b} |
| Ser | 267.01 (+1) | 13.25 | 1.3 |
| Gly | 237.02 (+1) | 13.83 | 1.8 |
| Asp | 295.00 (+1) | 14.18 | 4.2 |
| Arg | 336.05 (+1) | 14.64 | 2.3 |
| Glu | 309.01 (+1) | 16.34 | 3.3 |
| Pro | 277.04 (+1) | 16.67 | 4.5 |
| Thr | 281.03 (+1) | 17.16 | 3.8 |
| Ala | 251.04 (+1) | 17.55 | 1.6 |
| Tyr-OH | 343.01 (+1) | 19.21 | N.D |
| Tyr-NH₂ | 343.03 (+1) | 23.03 | 3.5 |
| (di-) Lys | 469.13 (+1) | 23.31 | 2.4 |
| | 235.07 (+2) | | |
| Val | 279.05 (+1) | 23.64 | 2.6 |
| (di-) Cyn | 562.01 (+1) | 23.84 | 4.4 |
| | 281.51 (+2) | | |
| Met | 311.01 (+1) | 24.00 | 4.6 |
| Tyr-OH, —NH₂ | 504.09 (+1) | 25.15 | N.D |
| | 252.55 (+2) | | |
| Ile | 293.05 (+1) | 25.63 | 1.1 |
| Leu | 293.05 (+1) | 26.08 | 1.0 |
| Phe | 327.02 (+1) | 26.52 | 1.3 |
| Trp | 366.02 (+1) | 26.88 | 1.9 |

^{a}From UV absorbance at 205 nm
^{b}N.D. = not detected

Reproducibility of derivatization was studied by analyzing the standard mixture ten times. Concentration was determined using peak areas derived from absorbance detection. The results of these analyses are presented in Table 2 as relative standard derivations. Reproducibility was generally better than 5%.

TABLE 2
Comparison of relative MS signal intensity for amino
acids derivatized with different reagents^{a}

| | Derivatizing reagent | | | | | |
|---|---|---|---|---|---|---|
| Amino | C₁-NA- | C₂-NA- | C₃-NA- | C₄-NA- | | BA-¹³C₆- |
| acid | NHS | NHS | NHS | NHS | NA-NHS | NHS |
| Glu | N.D.^{b} | 1.6 | 3.1 | 3.3 | 1.0 | 0.11 |
| Lys | 2.3 | 3.3 | 5.0 | 6.3 | 1.0 | 0.96 |
| Trp | 2.5 | 3.9 | 4.8 | 12.5 | 1.0 | 0.15 |

^{a}Data were from two sets of experiments. Signal intensities were normalized to NA-NHS = 1.0
^{b}N.D. = not detected

Derivative stability was examined by measuring UV peak-area in reversed phase chromatograms at 205 nm over a week. Amino acids acylated on primary amine groups with C₃-NA were found to be stable for a week at 4° C. or less. For Met and Cyn, peak areas could decrease by up to 20% in a week of storage at ambient temperature. Cyn degradation was the result of disulfide bond cleavage with the formation of Cys (m/z 308.1(+1)). Cys elutes between Asp and Arg in RPC. Degradation products for Met were not identified. Tyr O-acylation is also a problem. Generally it would be recommended to remove O-acylation by hydrolysis before either analysis or storage before analysis. Hydrolysis of the O-acylated Tyr derivative accelerates with increasing pH. The O-acylated Tyr derivative is easily hydrolyzed by addition of hydroxylamine to achieve a slightly basic pH. Thus, hydroxylamine treatment of samples before analysis is preferred.

Reversed phase chromatography. Hydrophilic amino acids do not interact with the hydrophobic stationary phase of an RPC column, typically eluting in the column void volume. This problem was circumvented by increasing the hydrophobicity and concomitantly the RPC retention time of amino acids through derivatization. In one example, this can be achieved by adding alkyl groups to nicotinic acid. By varying the aliphatic chain length in Cₙ-NA derivatizing agents it is possible to manipulate the retention times of amino acids widely in the reversed phase chromatogram. Extending the aliphatic chain length of Cₙ-NA also increases detection sensitivity.

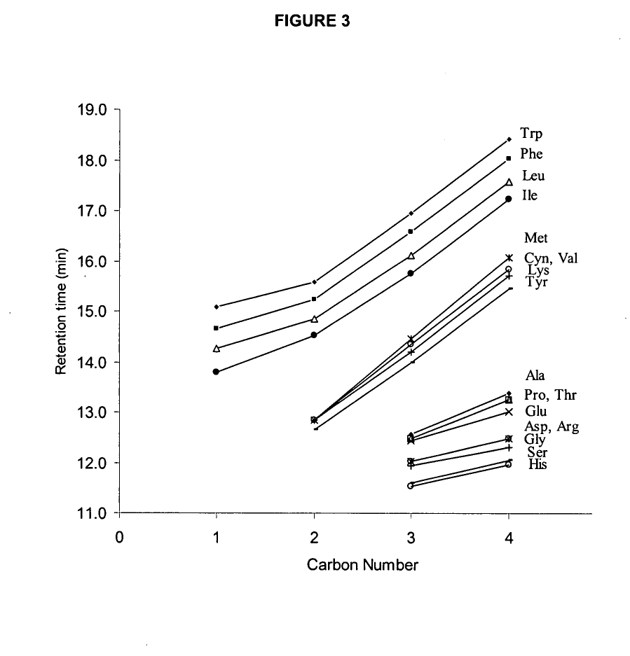

It is seen in FIG. 3 that the RPC retention time of even the most hydrophilic amino acids could be increased to greater than 10 minutes by derivatization. FIG. 3 is a graph showing the effects of alkyl chain length on retention time of derivatized amino acids. The following buffers were used: buffer A, consisting of 99.9% H₂O+0.1% TFA; buffer B, consisting of 95% ACN+0.1%TFA+4.9% H₂O. The elution protocol was: 0-5 minutes, 100% A; 5-30 minutes, 100% A-100% B. With the C₁-NA-NHS reagent only, Ile, Leu, Phe and Trp were detected. This reagent did not increase hydrophobility of the hydrophilic amino acids sufficiently to retain them beyond the trailing solvent peak. As the alkyl chain length was increased to 3, all 18 amino acids were adequately retained to be detected. C₄-NA-NHS was used in subsequent reactions because detection sensitivity was higher with C₄-NA derivatization.

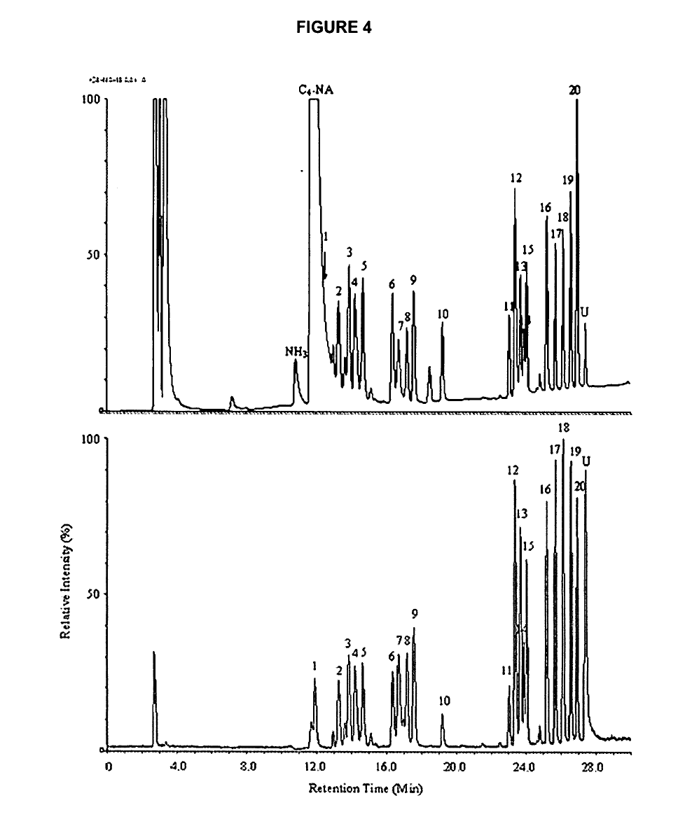

The RPC separation of 18 C₄-NA derivatized amino acids within 27 minutes is seen in FIG. 4. The derivatization time in this case was 1 minute. The upper trace was obtained with photodiode array detection from 200 to 400 nm. The lower trace was obtained by extraction ion monitoring from m/z 190-600. Elution of the RPC columns was achieved with a two-solvent gradient. Buffer A was 0.05% TFA in water while buffer B contained 0.05% TFA in ACN. The gradient protocol was as follows: 0-4 minutes, 100% A; 4-15 minutes, 0% B-10% B; 15-20 minutes, 10% B-20% B; and 20-30 minutes, 20% B-50% B. Peak identification is as follows: 1, His; 2, Ser; 3, Gly; 4, Asp; 5, Arg; 6, Glu; 7, Pro; 8, Thr; 9, Ala; 10, Tyr-OH; 11, Tyr-NH₂; 12, Bis-Lys; 13, Val; 14, Cyn; 15, Met; 16, Bis-Tyr; 17, Ile; 18, Leu; 19, Phe; 20, Trp; U, unknown.

Peaks were assigned based on their unique m/z values except for C₄-NA-Ile and C₄-NA-Leu, which have identical masses and showed the same singly charged ion at m/z 293.05(+1). They were distinguishable by both their chromatographic retention times and tandem mass spectra. C₄-NA-His was obscured in the photo diode array (PDA) trace by C₄-NA arising from the hydrolysis of C₄-NA-NHS. However, it was easily recognized in the extracted total ion chromatogram (TIC trace) when the m/z range was set for 190 to 600. However, there is a penalty from excluding ions under m/z 190. Although C₄-NA appears at m/z 180.0 and is eliminated from spectra, ammonia is also derivatized under these conditions and gives a distinct chromatographic peak. Thus this product is excluded as well. In addition to N-acylation, O-acylation of tyrosine (peak 10) and bis-tyrosine (peak 16) was observed with a short derivatization time of one minute (FIG. 4).

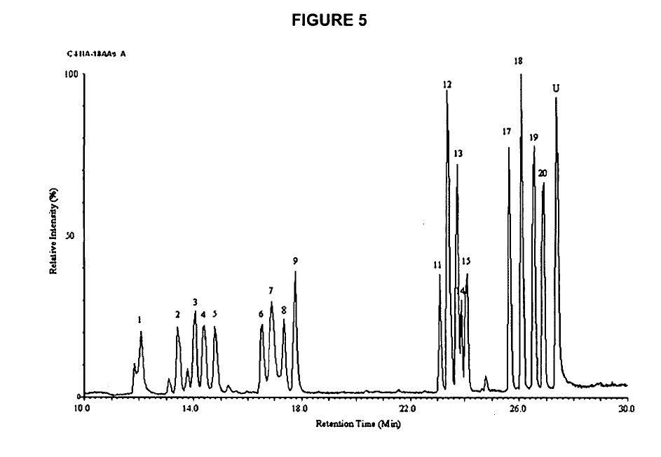

FIG. 5 is a graph showing the extracted total ion chromatogram from 18 amino acids derivatized with C₄-NA-NHS using 10-minute incubation time. Detection was achieved by monitoring the m/z 190-600 range. Separation conditions and peak identification are the same as in FIG. 4. As the derivatization time was increased from 1 to 10 minutes, it is shown in FIG. 5 that these peaks are no longer present. Although phenolic hydroxyl groups derivatize rapidly, they are not stable in water and hydrolyze during a 10 minute incubation.

One of the advantages of Cₙ-NA derivatization is that it also facilitates detection by UV absorbance through the addition of the aromatic ring in nicotinic acid. As shown in FIG. 4, the UV response of non-aromatic amino acids was similar with the exception of lysine (Lys) and cystine (Cyn). These amino acids were labeled by Cₙ-NA-NHS twice and thus have one more nicotinic acid residue than the other non-aromatic amino acids. Nicotinic acid residues show weak absorbance at 265 nm and absorb strongly below 210 nm.

Enhancement of ionization. The requisite acylation of amino groups in amino acids to achieve derivatization reduces their charge. Concomitantly, this is expected to decrease ionization efficiency in positive ionization mode ESI-MS. The impact of charge on ionization efficiency of derivatized amino acids was examined using nicotinoate and benzoate derivatized Glu, Try, and Lys. ¹³C₆ isotopically coded benzoyl derivatives (BA-¹³C₆) of Glu, Trp and Lys were prepared using methods described in the literature (Julka and Regnier, 2004, Anal. Chem. 76: 5799-5806). Use of BA-¹³C₆ was necessary because benzoate and nicotinoate vary by only 1 atomic mass unit. Equal amounts of these three amino acids were derivatized individually with ¹³C₆-BA-NHS and NA-NHS, respectively and examined individually by RPC. Peaks of analyte were collected, dried and redissolved in 50% methanol+49% H₂O+1% acetic acid (v/v/v). Equal aliquots of the six derivatives were mixed with the exception of BA-¹³C₆-Glu, which was added at five times the volume of the others. An ESI-MS analysis of the mixture was performed as described in the Experimental Section. Relative differences in detection sensitivity are summarized in Table 2. Derivatization with nicotinic acid was seen to enhance detection sensitivity with Glu and Try, but not Lys. This is attributed to the ionization of nitrogen in the pyridine ring.

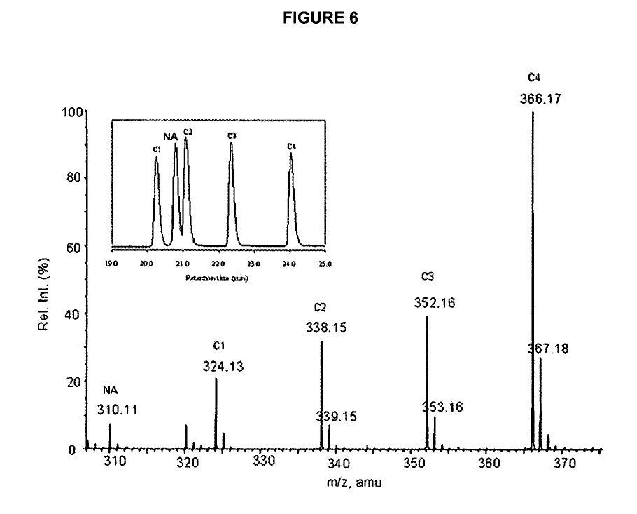

A more dramatic enhancement of sensitivity was observed for the amino acids derivatized through permanently charged and alkylated derivatizing reagents C₁₋₄-NA-NHS. The C₁₋₄-NA-Trp derivative mixture along with NA-Trp was prepared and examined by ESI-MS. Although UV absorbance of the C₁₋₄-NA-Trp derivatives was very similar, response in ESI-MS was substantially larger with the longer alkyl chain length derivatives (FIG. 6). FIG. 6 is a graph showing the mass spectra of NA- and C₁₋₄-NA derivatives of tryptophan. The insert is the reversed phase chromatogram of the NA- and C₁₋₄-NA derivatives of tryptophan with UV detection at 204 nm. All derivatives were of the same concentration. Compared to nicotinoate derivatized tryptophan (NA-Trp), C₄-NA-Trp gave 12.5 times greater MS signal intensity. Comparisons were also made with Glu and Lys (Table 2). Although the degree of enhancement was not as great with these amino acids, even with Glu signal intensity was more than 3 times larger.

The results in Table 2 show that increasing charge in the pyridine ring through quaternization enhances sensitivity as seen by the difference in response between NA-AA and C₁-NA-M. This phenomenon has also been noted in the literature with peptides (Ren et al., 2004, Anal. Chem. 76: 4522-4530), but this doesn't explain how longer alkyl chains further enhances sensitivity. During the ionization process in ESI-MS, species that are both hydrophobic and cationic migrate to the droplet surface. When present at high concentration, they even suppress the ionization of less hydrophobic cationic species by forcing them away from the droplet surface. In view of the fact that ionization is thought to occur from droplet surfaces in ESI-MS, this process has been offered as an explanation of ion suppression. Based on this model of ESI-MS, it is likely that lengthening the alkyl chain in C₁₋₄-NA-AA causes the derivatized amino acid to have more surfactant like properties. This in turn increases AA concentration at droplet surfaces in ESI-MS and enhances ionization. The net effect is that in comparison to BA-¹³C₆ derivatives, C₄-NA derivatives of Glu, Lys and Trp showed 30, 6, and 83 times greater ESI-MS signal intensity, respectively.

Comparative quantification through deuterium labeling. In vitro stable isotope coding is now widely used in proteomics for quantitative comparisons of peptide and protein concentration between samples. The most successful of these approaches is to acylate α- and ε-amino groups of polypeptides in a sample with a reagent that allows global coding of all primary amine containing species according to sample origin. When these differentially coded samples are then mixed and analyzed by ESI-MS the spectra contain doublet clusters of ions from the coded peptide isotopomers. The difference in mass between the doublets is simply the stable isotope mass differential between the isotopically coded labeling agents. The difference in peak area of the clusters is proportional to the relative concentration of the peptide in the two samples. The relative concentration of thousands of peptides can be determined in a single RPC-MS in this in vitro coding method with a relative standard deviation of roughly 6% (Julka and Regnier, 2004, J. Proteome Research 3: 350-363).

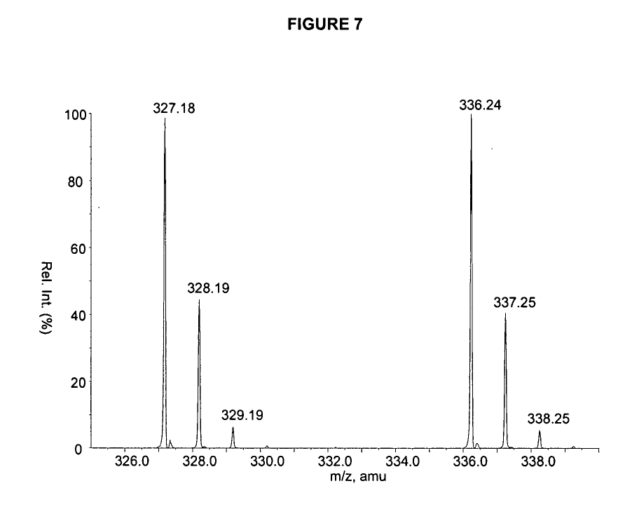

A heavy form of C₄-NA-NHS was synthesized by substituting D₉-n-butyl iodide for H₉-n-butyl iodide in the NA-NHS alkylation reaction, producing C₄D₉-NA-NHS, that is, 9 atomic mass units (amu) higher than the light form of the coding agent, C₄H₉-NA-NHS. Phenylalanine (Phe) was used for initial ESI-MS comparative quantification studies. C₄D₉-NA-Phe and C₄H₉-NA-Phe were mixed at concentration ratios varying over a 400 fold range in concentration. Isotope ratio analyses were performed by infusing samples into the ESI-MS through a nanospray inlet. As shown in FIG. 7, the characteristic double cluster of ions separated by 9 amu is seen in the spectrum of a 1:1 concentration ratio sample. FIG. 7 is a graph showing the mass spectra of C₄H₉-NA-Phe and C₄D₉-NA-Phe at a 1:1 molar ratio. The isotope ratio calibration curve was found to be linear over a 400 fold range of concentration with a linearity coefficient (r²) of 0.988. Thus, comparative quantification of amino acids by stable isotope coding is possible using ESI-MS.

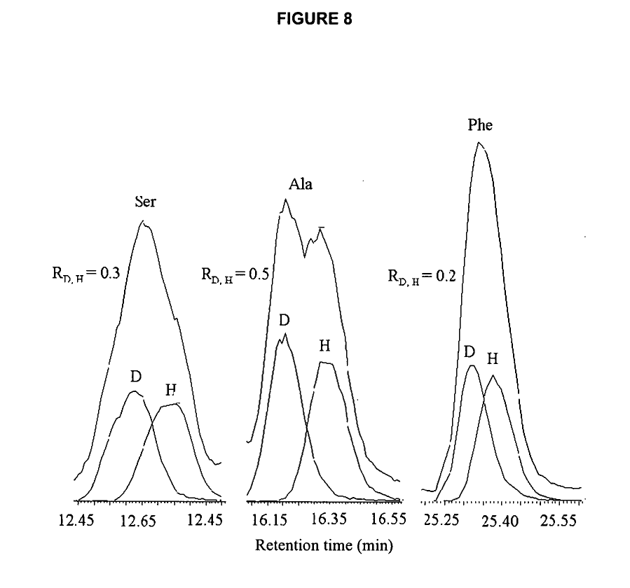

A more extensive study of comparative quantification was undertaken by differentially coding samples containing the entire set of amino acid standards. FIG. 8 is a graph showing the chromatographic isotope effects for three amino acids derivatized with C₄H₉-NA-NSH and C₄D₉-NA-NSH at the ratio of 1:1. D=deuterium labeled derivatives; H=non-labeled derivatives. R_{D,H} refers to resolution of the heavy and light isotope labeled derivatives. Experimental conditions are as in FIG. 5. These sample mixtures were prepared by splitting a mixture of amino acid standards into equal aliquots, i.e. the amino acid concentration ratio between the two samples was 1:1. When amino acids in these two samples were differentially coded with the C₄D₉-NA-NHS and C₄H₉-NA-NHS labeling agents, respectively and analyzed by RPC-MS, it was seen in multiple cases that the isotopomers of amino acids did not coelute (FIG. 8). This meant that isotope ratio analysis could only be achieved by comparing the chromatographic peak area of amino acid isotopomers obtained from extracted ion chromatograms constructed after the analysis of the mixture was finished. Real-time isotope ratio analysis is precluded in cases where amino acid isotopomers are partially resolved because the isotope ratio at any single point in time does not reflect that in the initial mixture. This chromatographic isotope effect is common with deuterated isotopomers. It has also been noted with peptide isotopomers that the chromatographic isotope effect can be circumvented by placing deuterium atoms in the coding agent beside a quaternary amine or by using ¹³C coding instead of ²H (Zhang et al., 2002, Anal. Chem. 74: 3662-3669). Stable isotope based quantification can be of value in metabolomics and other fields where it is the objective to compare the concentration of amino acids between samples.

RPC/ESI-MS Analysis. Optimization of the derivatization reaction was achieved using RPC on an Integral Micro-Analytical workstation with a 205 nm wavelength UV detector (Applied Biosystems, Framingham, Mass.). Amino acid derivatives were also purified with this system for direct MS analysis in some cases. RPC with electrospray ionization (ESI) mass spectral analysis of amino acid derivatives was carried out on a Waters Alliance system, equipped with MassLynx 4.0 software, a Waters 996 Photodiode Array Detector (PAD), and a Micromass Q-Tof micro mass spectrometer. The PAD was used in the scanning mode to detect analytes between 200 and 400 nm. Positive ion mass spectra were acquired in the continuous mode. Ionization was achieved with an electrospray voltage of 3 kV, cone voltage of +30 V, cone and desolvation gas flows of 5-8 and 500 L/h, respectively. The source block and desolvation temperatures were maintained at 150° C. and 350° C., respectively. All separations were performed on a low TFA Vydac analytical column (C18 Mass Spec, 4.6 mm×250 mm) using 15 μL of sample and gradient elution with two sets of mobile phases. Optimization of derivatization was achieved by eluting the RPC column initially with 5% ACN and 0.1% TFA in water for 5 minutes and then proceeding on in a 30 min linear gradient to 5% water and 0.1% TFA in ACN. General analyses were achieved with a second elution protocol using two different eluting solvents. Solvent A was composed of 0.05% TFA in water and solvent B contained 5% H₂O and 0.05% TFA in ACN. After 4 minutes of isocratic elution with solvent A an 11 minute linear gradient to 10% solvent B was initiated followed by a 5 minute linear gradient to 20% mobile phase B and finally a 10 minute linear gradient to 50% solvent B. The flow rate was 1 ml/min in all cases.

ESI-MS/MS Analysis. ESI-MS and ESI/MS/MS analysis were performed on an API QSTAR® Pulsar LC/MS/MS System (Applied Biosystems, Framingham, Mass.) equipped with an ESI ion source. Fractions from the integral LC were collected, dried, and reconstituted with CH₃OH/H₂O/acetic acid (50%/49%/1%), and injected into the ESI source by infusion at 5-10 μl/min. Typical settings for analysis in the positive mode of ESI were as follows: ionspray voltage, 5000; curtain gas, 20; ion source gas 1, 20; ion source gas 2, 0; declustering potential, 45; focusing potential, 220; declustering potential 2, 20.0. Collision energy was optimized to obtain maximum fragmentation for tandem MS.

Synthesis of 4-iodo-[2,5-dioxopyrrolidin-1-yl]butyrate. This reagent was synthesized according to the procedure by Taran et. al, 1998, J. Amer. Chem. Soc. 120: 3332-3339). 4-iodobutyric acid (1 g, 467 mmol), N-hydroxysuccinimide (0.54 mg, 4.69 mmol), and dicyclohexylcarbodiimide (0.96 g, 4.66 mmol) were dissolved in 10 mL of AcOEt. The reaction mixture was stirred at room temperature for 16 hours. The dicyclohexyl urea formed was removed by filtration over Celite, and the crude material was concentrated and chromatographed on silica gel (petroleum ether/AcOEt, 1/1). The resultant yellow powder was recrystalized from petroleum ether/EtOH (9/1, v/v) yielding activated ester 4-iodo-[2,5-dioxopyrrolidin-1-yl]butyrate.

Synthesis of [3-(2.5)-dioxopyrrolidin-1-yloxycarbonyl)-propyl]-dimethyloctylammonium (C₈-QAT). This reagent was synthesized according to the procedure by Taran et. al., 1998, J. Amer. Chem. Soc. 120: 3332-3339, with slight modifications. N,N-dimethyl-N-octylamine (280 mg, 1.8 mmol), activated ester 4-iodo-[2,5-dioxopyrrolidin-1-yl]butyrate (550 mg, 1.8 mmol), and silver trifluoromethanesulfonate (460 mg, 1.8 mmol) were dissolved in 2 mL of anhydrous acetone. The solution was stirred for 16 hours at room temperature. The silver iodide formed was removed by filtration over Celite, and the solvent was evaporated. The resultant orange oil was dissolved in 1 ml of ACN, and the final product was precipitated by addition of 10 ml of EtOAc.

Synthesis of (3-Carboxypropyl)-trimethylammonium chloride. (CH₃)N⁺(Cl)CH₂CH₂CH₂—(C═O)OH was prepared according to the method described by Sioma, 2003, MS Thesis, Purdue University. 10.0 g (0.097 mol) of 4-aminobutanopic acid, 48.0 g (0.497 mol) of KHCO₃ and 30 mL (0.482 mol) of CH₃I were stirred in 1 L of MeOH for 24 hours. The MeOH was removed under vacuum and the crude residue slurried with 200 mL of CHCl₃, then filtered and acidified with 50 mL of concentrated HCl. The H₂O was removed under vacuum and the remaining residue extracted with 400×2 mL of anhydrous acetone, concentrated under reduced pressure, slurried with 200 mL of THF and filtered to yield the final product.

Synthesis of [3-(2,5)-dioxopyrrolidin-1-yloxycarbonyl)-propyl]-trimethylammonium (QAT). This reagent was prepared according to the method of Sioma, 2003, MS Thesis, Purdue University. 13.2 g (0.073 mol) of (3-Carboxypropyl)-trimethylammonium chloride, 8.4 g (0.073 mol) of NHS and 16.5 g (0.080 mol) of DCC were dissolved in ACN. Once dissolved, the resulting solution was refrigerated for 24 hours. The formed 1,3-dicyclohexylurea was filtered off and the ACN was evaporated under vacuum. The crude residue was slurried with 200 mL of THF, filtered and recrystallized from ACN/THF to yield the final product.

Derivatization of model peptides with QAT and C₈-QAT reagent. Model peptides were dissolved in 50 mM HEPES pH 8.00 at a final concentration of 1 mg/ml. A 50-fold molar excess of each derivatization reagent was added individually to the peptide solutions and the reaction was allowed to proceed for 2 hours at room temperature. After the reaction was completed, N-hydroxylamine was added in excess, and the pH was adjusted to 11-12 with sodium hydroxide to hydrolyze esters. The reaction was allowed to proceed for 10 minutes, then the pH was adjusted back to 7-8 with 1 to 2 drop of glacial acetic acid.

Proteolysis. To denature, reduce and alkylate transferrin, urea and DTT were added to a final concentration of 6 M and 10 mM, respectively. Mixtures were incubated for one hour at 37° C., iodoacetamide was then added to a final concentration of 20 mM and the reaction was allowed to proceed for an additional 30 minutes at 4° C. Cysteine was then added to final concentration of 10 mM to quench extra iodoacetamide. Samples were diluted six-fold with 50 mM HEPES, pH 8.0, 10 mM MgCl₂, and 10 mM CaCl₂. Sequence grade trypsin was added (2%), and the reaction mixture was incubated at 37° C. for at least 8 hours. Proteolysis was stopped by adding TLCK (trypsin:TLCK ratio of 1:1 (w/w)).

Derivatization of model peptides with QAT and C₈-QAT reagent. A 50 fold molar excess of each derivatization reagent was added to the tryptic digest peptide and the reaction was allowed to proceed for 2 hours at room temperature. After the reaction was completed, N-hydroxylamine was added in excess and the pH was adjusted to 11-12 with sodium hydroxide to hydrolyze esters. The reaction was allowed to proceed for 10 minutes, then the pH was adjusted back to 7-8 with 1 to 2 drop of glacial acetic acid.

LC/MS analysis. Derivatized and non-derivatized model peptides as well as transferrin digest were separated on an Agilent Zorbax C₈ (0.3×150) and C₁₈ column (0.5×150 mm) using an Agilent 1100 series instrument (Agilent Technologies, Inc., Palo Alto, Calif.) at 4 μL/min. Solvent A was 0.01% TFA in deionized H₂O (dl H₂O) and solvent B was 95% ACN/0.01% TFA in dl H₂O. The flow from the column was directed to a Q-STAR workstation (Applied Biosystems, Framingham, Mass.) equipped with an ESI source. Flow from the HPLC was diverted to waste for 10 minutes after sample injection at 100% solvent A to remove salts, remaining derivatizing reagent APTA, and weakly adsorbed peptides. The Q-STAR was then reconnected and peptides were separated in a 15 minutes linear gradient (from 0% B to 60% B). MS spectra were obtained in the positive ion mode at a sampling rate of one spectrum per second.

LC separation of tryptic peptides. Peptides from the transferrin tryptic digest were separated on a Vydac C₁₈ column (4.6×250 mm) using a BioCAD 20 Micro-Analytical Workstation (Applied Biosystems, Framingham, Mass.) at 1 mumin. Solvent A was 0.1% TFA in deionized H₂O (dl H₂O) and solvent B was 95% ACN/0.1% TFA in dl H₂O. Peptides were separated in a 60-minute linear gradient (from 0% B to 60% B).

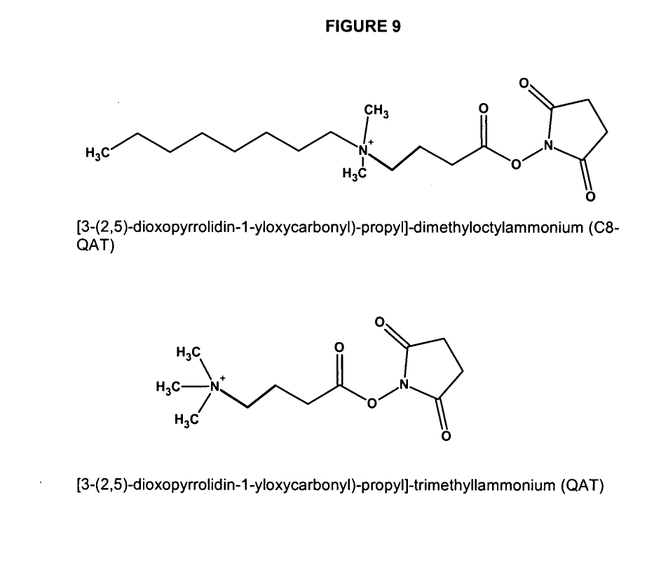

Derivatizing agent architecture. The strategy for enhancing electrospray ionization of peptides through derivatization was to use a derivatizing agent that would: 1) have both quaternary amine and alkyl groups, 2) N-acylate primary amines, 3) be pre-activated to form peptide bonds, and 4) acylate peptides in water. The reagent [3-(2,5)-dioxopyrrolidin-1-yloxycarbonyl)-propyl]-dimethyloctylammonium (FIG. 9) was developed to meet these criteria. FIG. 9 shows the structures of the QAT and C₈-QAT reagents. The C₈-QAT reagent is more hydrophobic than QAT and will convey greater hydrophobicity to labeled peptides. Peptides derivatized with this reagent are designated as having a C₈-quaternary amine tag (C₈-QAT). When activated with N-hydroxysuccinimide (NHS), this tagging agent is referred to as C₈-QAT-NHS. The synthesis of this derivatizing agent is similar to that of [3-(2,5)-dioxopyrrolidin-1-yloxycarbonyl)-propyl]-trimethylammonium (FIG. 9) which has been described by Sioma, 2003, MS Thesis, Purdue University. Peptides derivatized with this quaternary amine tag (QAT) have improved ionization efficiency (Sioma, 2003, MS Thesis, Purdue University).

Peptide derivatization. The C₈-QAT-NHS labeling agent was added to peptide mixtures in at least a 50-fold molar excess and incubated at room temperature overnight. The reaction was terminated by addition of excess N-hydroxylamine and the pH adjusted to 11-12 with a few drops of saturated sodium hydroxide solution. Hydrolysis of C₈-QAT-NHS and any O-acylation products was achieved in 10 minutes after the addition of base. The pH was then adjusted back to 7-8 with 1 to 2 drops of glacial acetic acid. Reactivity of the C₈-QAT-NHS was found to be the same as that of the QAT-NHS reagent.

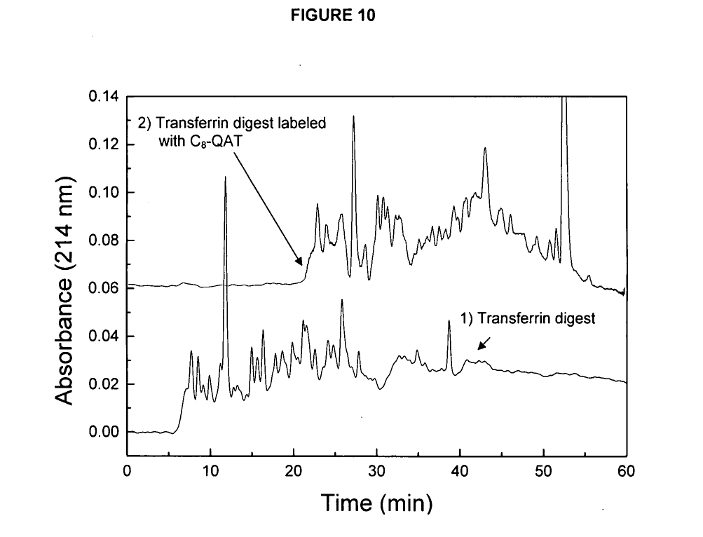

The effect of C₈-QAT labeling on reversed phase chromatographic retention time. The impact of C₈-QAT derivatization on peptide retention in RPC was studied using a transferrin tryptic digest. Native peptides and those tagged with C₈-QAT were prepared and fractionated in separate runs on a Vydac C₁₈ column using a 60 minute linear gradient starting from solvent A to 60% solvent B. FIG. 10 shows overlaid chromatograms of the labeled and unlabeled tryptic peptide digests. C₈-QAT labeling induced about a 20 minute increase in the retention time of peptides. The relative increase in retention of small hydrophilic peptides was greater than that of larger hydrophobic peptides.

FIG. 10 is a graph depicting reversed-phase chromatograms for 1) unlabeled and 2) C₈-QAT labeled transferrin digest separated on a C₁₈ column. The digests were separated on a Vydac C₁₈ column using a 60 minute gradient from 99.5% buffer A (0.01% TFA in deionized H₂O (dl H₂O)) to 60% buffer B (95% ACN/0.01% TFA in dl H₂O). There is a 15 minutes delay in retention time caused by increased hydrophobicity of the labeled peptides. The labeled digest also shows a higher level of complexity and longer gradient elution due to retention of very hydrophilic peptides that did not retain before labeling. The labeling reagent peaks were removed by subtraction of labeling reagent only chromatogram from the labeled digests.

Analyte retention in reversed phase chromatography (RPC) is generally accepted to be due to a solvophobic effect driven by the surface tension of a polar mobile phase. Polar solvents force hydrophobic molecules to interact with molecules that are similarly hydrophobic to minimize their contact area with the solvent. Although peptides have a large number of polar groups, the side chains of many amino acids in peptides are hydrophobic. The solvophobicity of these hydrophobic groups in polar mobile phases drives peptides to interact with the solvophobic surface of RPC columns. But not all peptides are retained by RPC columns. Those that are not retained lack sufficient hydrophobicity to interact with the stationary phase. As observed, the hydrophobic C₈-QAT would be expected to increase the retention of a small hydrophilic peptide in RPC more than that of a large hydrophobic peptide that already interacts strongly with the stationary phase.

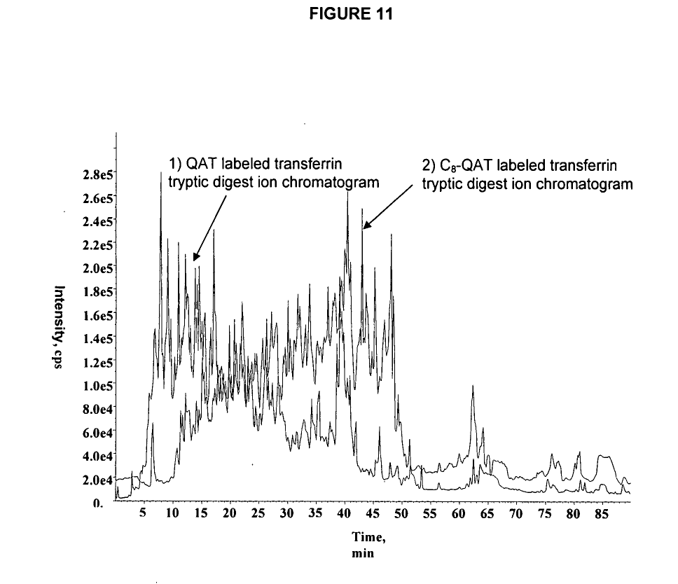

Although increasing the retention of hydrophilic peptides beyond the solvent front is desirable, a 20 minute increase in retention is greater than needed. The QAT and C₈-QAT derivatized transferrin digests were also examined with an octyl silane (C₈ Zorbax) column (FIG. 11). FIG. 11 is a graph showing ion chromatograms for: 1) QAT-labeled and 2) C₈-QAT labeled transferrin digests separated on a C₈ reversed-phase column. Separation of transferrin digests on a less hydrophobic reversed-phase column such as C₈, allows the hydrophobic effect to become more dominant. The retention time delay as a result of C₈-QAT labeling is reduced to 5 minutes and elution of C₈-QAT labeled peptides at lower organic percentage, enhances the hydrophobic effect. Some QAT labeled peptides (5%) do not retain on C₈ column, but overall there is 1.25-fold increase in ionization efficiency. The C₈-QAT labeled peptides are also distributed more evenly across the chromatogram. Decrease in local concentration of peptides in droplets also decreases the ionization suppression.

Comparing the chromatograms in FIGS. 10 and 11, it is seen that retention of peptides tagged with the C₈-QAT reagent is substantially shorter on the octyl (C₈) than the octadecyl (C₁₈) RPC column. The reduced hydrophobicity of the C₈ Zorbax column compensates for the increased hydrophobicity of peptides derivatized with the C₈-QAT reagent. Directing the non-retained peak from the C₈ column into a C₁₈ column showed that 5% of the peptides captured by the C₁₈ column were not retained on the C₈ column. Peptides not retained by the C₈ column are generally small hydrophilic peptides of minimal value in protein identification.

The effect of C₈-QAT labeling on electrospray ionization of model peptides. Tagging peptides with C₈-QAT modifies the peptides in two important ways that might impact electrospray ionization. One is the introduction of a quaternary amine, giving them a permanent positive charge. The second is to make them more hydrophobic. The relative contribution of quaternization was studied by derivatizing model peptides with the QAT reagent. As noted in FIG. 9, the QAT and C₈-QAT reagents are identical in structure with the exception of the substitution of an octyl-group for a methyl group on the quaternary amine. Some peptides tagged with the (QAT) reagent experience a moderate increase in electrospray ionization efficiency (Sioma, 2003, MS Thesis, Purdue University).

The relative ionization of native, QAT labeled, and C₈-QAT labeled peptides was studied using 11 model peptides varying in size, hydrophilic to hydrophobic amino acid ratio, and number of cationic amino acids (Table 3). Hydrophobicity (HP) of native peptides was calculated using relative hydrophobicity of their amino acids. All the model peptides were labeled separately with both the QAT and C₈-QAT reagents and mixed with native peptides in a 1:1:1 ratio. The mixture (5 μL or 20 μL injection volume, as indicated in the text) was separated on a Zorbax C₁₈ column using a 60 minute gradient starting from 99.5% buffer A (0.01% TFA in deionized H₂O (dl H₂O)) to 60% buffer B (95% ACN/0.01% TFA in dl H₂O). Relative ionization (Rᵢ) of peptides was calculated using the formula

$${R}_{i} = \frac{\sum {A}_{i}^{+ n}}{\sum {A}_{native}^{+ n}}$$

where ΣAₙₐₜᵢᵥₑ⁺ⁿ is the sum of area under the curves of all charge states of the native peptide and ΣAᵢ⁺ⁿ is the sum of area under the curves for all the charge states of the peptide for which Rᵢ is being calculated (peptide i). Since Rᵢ is normalized against a native peptide, the value of Rᵢ for the native peptide is always 1.

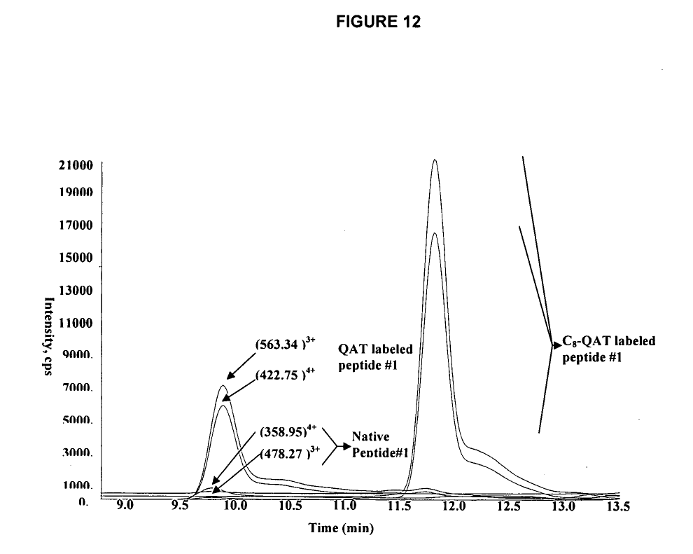

FIG. 12 is a graph showing the extracted ion chromatograms for native, QAT and C₈-QAT labeled model peptide Ac-Gln-Lys-Arg-Pro-Ser-Gln-Arg-Ser-Lys-Tyr-Leu-OH. All the charge states for different forms of the peptide were extracted from the total ion chromatogram. The total ion chromatogram was obtained by separation of a mixture of native, QAT and C₈-QAT labeled peptide mixed in 1:1:1 ratio on a C₁₈ column. The area under the peak for each charge state of each labeled peptides was calculated and then summed. The summation of peak areas of all charge states of a peptide form was used as a representative of total ionization for that peptide.

Table 3 shows a list of model peptides used to study the effect of QAT and C₈-QAT labeling on ionization efficiency. All peptides were labeled with QAT and C₈-QAT. Native, QAT labeled and C₈-QAT labeled forms of each peptide were mixed in 1:1:1 ratio and separated on a C₁₈ column. The injection volume was 5 μL in all cases. The areas under the peak for all charge states of each peptide were summed and used as a total ionization measure. The ratios were calculated by dividing the total peak areas for each peptide by the total peak area of the native peptide.

It is seen in Table 3 that, for the peptide Ac-Gln-Lys-Arg-Pro-Ser-Gln-Arg-Ser-Lys-Tyr-Leu-OH, the Rᵢ for the C₈-QAT peptide is 20 while Rᵢ for the QAT derivatized form of the peptide is 12. This peptide has two primary amine groups and was derivatized twice. A similar behavior is seen in the peptide H-Tyr-Gly-Gly-Phe-Met-Lys-OH, which is labeled twice. Ionization efficiency of the C₈-QAT derivative is 33 times that of the native peptide and 5 times greater than the QAT derivative. The most dramatic differences in ionization efficiency were seen with the peptides H-Ala-Gly-Gly-OH and H-Ala-Met-OH which were singly labeled. Ionization of native H-Ala-Gly-Gly-OH was not detectable relative to the C₈-QAT derivative while that of native H-Ala-Met-OH was 125 times lower than that of the C₈-QAT tagged peptide. Ionization efficiency of the C₈-QAT derivative was also much larger than that of the QAT derivative in the case of these peptides. The small peptide H-Ala-Ser-OH also experienced a 20-fold increase in ionization efficiency after C₈-QAT derivatization. Although the QAT tag increased the ionization efficiency of these peptides, derivatization with the C₈-QAT group clearly had a much more dramatic impact on ionization efficiency of these peptides.

In contrast, derivatization of the peptides H-Asp-Arg-Val-Tyr-Ile-His-Pro-Phe-His-Leu-OH, H-Cys-Asp-Pro-Gly-Tyr-Ile-Gly-Ser-Arg-OH, H-Ser-Tyr-Ser-Met-Glu-His-Phe-Arg-Trp-Gly-OH, and H-Ala-Phe-Pro-Leu-Glu-Phe-OH with the QAT or C₈-QAT labels either decreased ionization efficiency slightly or had little effect. The single derivatizable primary amine in these peptides was at the amino terminus. Derivatization had an equally small impact on the ionization efficiency of H-Pro-His-Pro-Phe-His-Phe-His-Phe-Phe-Val-Tyr-Lys-OH which was singly labeled at the C-terminal lysine.

TABLE 3
List of model peptides used to study the effect of QAT and C₈-QAT labeling on ionization efficiency

| | | | | | | | | Rᵢ based ratio |
|---|---|---|---|---|---|---|---|---|
| | Peptide | Native | area | QAT | area | C₈-QAT | area | Native:QAT:C₈-QAT |
| 1 | Ac-Gln-Lys- | (715.89)²⁺ | 0 | (844.50)²⁺ | 203 | (942.11)²⁺ | 0 | 1:12:20 |
| | Arg-Pro-Ser- | (477.60)³⁺ | 260 | (563.34)³⁺ | 1364 | (628.41)³⁺ | 2519 | |
| | Gln-Arg-Ser- | (358.95)⁴⁺ | 0 | (422.75)⁴⁺ | 1663 | (471.56)⁴⁺ | 2471 | |
| | Lys-Tyr-Leu- | | | | | | | |
| | OH (HP: −24.5) | | | | | | | |
| 2 | H-Asp-Arg- | (1296.69)¹⁺ | 2362 | (1423.78)¹⁺ | 25 | (1521.89)¹⁺ | 24 | 1:0.82:0.21 |
| | Val-Tyr-Ile- | (648.89)²⁺ | 30491 | (712.39)²⁺ | 18204 | (761.45)²⁺ | 14058 | |
| | His-Pro-Phe- | (432.89)³⁺ | 25886 | (475.26)³⁺ | 30599 | (507.49)³⁺ | 5175 | |
| | His-Leu-OH | | | | | | | |
| | (HP: −1.3) | | | | | | | |
| 3 | H-Ala-Phe- | (723.37)¹⁺ | 26652 | (850.47)¹⁺ | 23236 | (948.58)¹⁺ | 11548 | 1:1:0.56 |
| | Pro-Leu-Glu- | (362.19)²⁺ | 724 | (425.73)²⁺ | 3190 | (474.79)²⁺ | 3372 | |
| | Phe-OH (HP: | (241.79)³⁺ | 0 | (284.16)³⁺ | 0 | (316.86)³⁺ | 296 | |
| | 6.1) | | | | | | | |
| 4 | H-Ser-Tyr- | (1299.55)¹⁺ | 1470 | (1426.65)¹⁺ | 208 | (1524.76)¹⁺ | 111 | 1:1.91:0.83 |
| | Ser-Met-Glu- | (650.28)²⁺ | 21985 | (713.83)²⁺ | 15123 | (762.88)²⁺ | 11110 | |
| | His-Phe-Arg- | (433.85)³⁺ | 6631 | (476.22)³⁺ | 42773 | (508.59)³⁺ | 14162 | |
| | Trp-Gly-OH | | | | | | | |
| | (HP: −10.7) | | | | | | | |
| 5 | H-Cys-Asp- | (967.43)¹⁺ | 5797 | (1094.53)¹⁺ | 686 | (1192.64)¹⁺ | 50 | 1:0.14:0.34 |
| | Pro-Gly-Tyr- | (484.21)²⁺ | 4160 | (554.27)²⁺ | 893 | (596.82)²⁺ | 3691 | |
| | Ile-Gly-Ser- | (323.14)³⁺ | 0 | (369.84)³⁺ | 0 | (398.22)³⁺ | 0 | |
| | Arg-OH (HP: −5.1) | | | | | | | |
| 6 | H-Tyr-Gly- | (701.32)¹⁺ | 0 | (957.53)¹⁺ | 339 | (1153.75)¹⁺ | 28938 | 1:6.67:33.3 |
| | Gly-Phe-Met- | (351.16)²⁺ | 1656 | (479.27)²⁺ | 9766 | (576.87)²⁺ | 28603 | |
| | Lys-OH (HP: −1.3) | (234.78)³⁺ | 0 | (319.51)³⁺ | 0 | (384.92)³⁺ | 0 | |
| 7 | H-Pro-His- | (1318.67)¹⁺ | 771 | (1445.77)¹⁺ | 53 | (1543.88)¹⁺ | 46 | 1:2:1 |
| | Pro-Phe-His- | (659.84)²⁺ | 9579 | (723.39)²⁺ | 4056 | (772.44)²⁺ | 5569 | |
| | Phe-His-Phe- | (440.22)³⁺ | 12440 | (482.59)³⁺ | 6724 | (515.29)³⁺ | 17173 | |
| | Phe-Val-Tyr- | | | | | | | |
| | Lys-OH (HP: −2.6) | | | | | | | |
| 8 | H-Ala-Leu- | (259.15)¹⁺ | 3082 | (387.26)¹⁺ | 2687 | (485.37)¹⁺ | 47781 | 1:1:16.67 |
| | Gly-OH (HP: 5.2) | (130.07)²⁺ | 0 | (194.14)²⁺ | 0 | (243.18)²⁺ | 0 | |
| 9 | H-Ala-Gly- | (203.09)¹⁺ | 0 | (331.19)¹⁺ | 3170 | (429.31)¹⁺ | 2781 | 1:16.67 |
| | Gly-OH | (102.16)²⁺ | 0 | (166.10)²⁺ | 0 | (215.15)²⁺ | 10251 | |
| | (HP: 1) | | | | | | | |
| 10 | H-Ala-Met- | (220.08)¹⁺ | 355 | (348.19)¹⁺ | 10989 | (446.30)¹⁺ | 43094 | 1:31.25:125 |
| | OH (HP: 3.7) | (110.54)²⁺ | 0 | (174.60)²⁺ | 0 | (223.65²⁺ | 0 | |
| 11 | H-Ala-Ser-OH | (176.07)¹⁺ | 155 | (304.18)¹⁺ | 2156 | (402.29)¹⁺ | 2304 | 1:20:20 |
| | (HP: 1) | (130.07)²⁺ | 0 | (152.50)²⁺ | 0 | (201.65)²⁺ | 0 | |

When the same 11 model peptides (shown in Table 3) along with the QAT and C₈-QAT derivatives were examined at 4 times higher concentration, a different picture of ionization efficiency emerged (Table 4). Table 4 shows a list of model peptides used to study the effect of QAT and C₈-QAT labeling on ionization efficiency. All peptides were labeled with QAT and C₈-QAT. Native, QAT labeled and C₈-QAT labeled forms of each peptide were mixed in 1:1:1 ratio and separated on a C₁₈ column. The injection volume was 20 μL in all cases (4 times the amount used in Table 3). The areas under the peak for all charge states of each peptide were summed and used as a total ionization measure. The ratios were calculated by dividing the total peak areas for each peptide by the total peak area of the native peptide (Rᵢ). In higher concentration all C₈-QAT labeled peptides show higher ionization efficiency than other forms of the same peptide (native and QAT labeled).

Increases in ionization efficiency of the peptides Ac-Gln-Lys-Arg-Pro-Ser-Gln-Arg-Ser-Lys-Tyr-Leu-OH and H-Tyr-Gly-Gly-Phe-Met-Lys-OH after C₈-QAT derivatization were 7-fold and 20-fold, respectively at the higher concentration, as opposed to 20-fold and 33-fold at the lower concentration. There was also a 1-fold to 7-fold increase in the ionization efficiency of other peptides greater than 500 Daltons in the mixture. Only with peptides of lower than 500 Daltons was there an increase in ionization efficiency greater than 300.

TABLE 4
List of model peptides used to study the effect of QAT and C₈-QAT labeling on ionization efficiency

| | | | | | | | | Rᵢ based ratio |
|---|---|---|---|---|---|---|---|---|
| | Peptide | Native | area | QAT | area | C₈-QAT | area | Native:QAT:C₈-QAT |
| 1 | Ac-Gln-Lys-Arg- | (715.89)²⁺ | 0 | (844.50)²⁺ | 0 | (942.11)²⁺ | 127 | 1:2.85:7.14 |
| | Pro-Ser-Gln- | (478.27)³⁺ | 591 | (563.34)³⁺ | 3798 | (628.41)³⁺ | 7621 | |
| | Arg-Ser-Lys-Tyr- | (358.95)⁴⁺ | 1587 | (422.75)⁴⁺ | 2189 | (471.56)⁴⁺ | 7154 | |
| | Leu-OH | | | | | | | |
| | (HP: −24.5) | | | | | | | |
| 2 | H-Asp-Arg-Val- | (1296.69)¹⁺ | 305 | (1423.78)¹⁺ | 59 | (1521.89)¹⁺ | 78 | 1:1:2 |
| | Tyr-Ile-His-Pro- | (648.89)²⁺ | 7259 | (712.39)²⁺ | 3845 | (761.45)²⁺ | 13672 | |
| | Phe-His-Leu-OH | (432.89)³⁺ | 5865 | (475.26)³⁺ | 6388 | (507.49)³⁺ | 11320 | |
| | (HP: −1.3) | | | | | | | |
| 3 | H-Ala-Phe-Pro- | (723.37)¹⁺ | 32622 | (850.47)¹⁺ | 29211 | (948.58)¹⁺ | 19766 | 1:1:1 |
| | Leu-Glu-Phe-OH | (362.19)²⁺ | 0 | (425.73)²⁺ | 3661 | (474.79)²⁺ | 13012 | |
| | (HP: 6.1) | (241.79)³⁺ | 559 | (284.16)³⁺ | 138 | (316.86)³⁺ | 1076 | |
| 4 | H-Ser-Tyr-Ser- | (1299.55)¹⁺ | 43 | (1426.65)¹⁺ | 20 | (1524.76)¹⁺ | 229 | 1:1.46:7.69 |
| | Met-Glu-His- | (650.28)²⁺ | 2286 | (713.83)²⁺ | 2525 | (762.88)²⁺ | 22748 | |
| | Phe-Arg-Trp- | (433.85)³⁺ | 2848 | (476.22)³⁺ | 4787 | (508.59)³⁺ | 15638 | |
| | Gly-OH | | | | | | | |
| | (HP: −10.7) | | | | | | | |
| 5 | H-Cys-Asp-Pro- | (967.43)¹⁺ | 2310 | (1094.53)¹⁺ | 1121 | (1192.64)¹⁺ | 928 | 1:0.2:2 |
| | Gly-Tyr-Ile-Gly- | (484.21)²⁺ | 3148 | (554.27)²⁺ | 0 | (596.82)²⁺ | 9955 | |
| | Ser-Arg-OH | (323.14)³⁺ | 0 | (369.84)³⁺ | 0 | (398.22)³⁺ | 0 | |
| | (HP: −5.1) | | | | | | | |
| 6 | H-Tyr-Gly-Gly- | (701.32)¹⁺ | 0 | (957.53)¹⁺ | 668 | (1153.75)¹⁺ | 576 | 1:6.8:20 |
| | Phe-Met-Lys-OH | (351.16)²⁺ | 1316 | (479.27)²⁺ | 8674 | (576.87)²⁺ | 26563 | |
| | (HP: −1.3) | (234.78)³⁺ | 0 | (319.51)³⁺ | 00 | (384.92)³⁺ | 0 | |
| 7 | H-Pro-His-Pro- | (1318.67)¹⁺ | 188 | (1445.)¹⁺ | 129 | (1543.88)¹⁺ | 0 | 1:0.33:6.67 |
| | Phe-His-Phe- | (659.84)²⁺ | 3958 | (723.39)²⁺ | 921 | (772.44)²⁺ | 9056 | |
| | His-Phe-Phe- | (440.22)³⁺ | 2437 | (482.59)³⁺ | 1112 | (515.29)³⁺ | 33390 | |
| | Val-Tyr-Lys-OH | | | | | | | |
| | (HP: −2.6) | | | | | | | |
| 8 | H-Ala-Leu-Gly- | (259.15)¹⁺ | 129 | (387.26)¹⁺ | 2002 | (485.37)¹⁺ | 50151 | 1:13.3:333 |
| | OH (HP: 5.2) | (130.07)²⁺ | 0 | (194.14)²⁺ | 0 | (243.18)²⁺ | 0 | |
| 9 | H-Ala-Gly-Gly- | (203.09)¹⁺ | 0 | (331.19)¹⁺ | 0 | (429.31)¹⁺ | 6598 | ∞ |
| | OH (HP: 1) | (102.16)²⁺ | 0 | (166.10)²⁺ | 0 | (215.15)²⁺ | 0 | |
| 10 | H-Ala-Met-OH | (220.08)¹⁺ | 0 | (348.19)¹⁺ | 0 | (446.30)¹⁺ | 12507 | ∞ |
| | (HP: 3.7) | (110.54)²⁺ | 0 | (174.60)²⁺ | 0 | (223.65²⁺ | 0 | |
| 11 | H-Ala-Ser-OH | (176.07)¹⁺ | 0 | (304.18)¹⁺ | 189 | (402.29)¹⁺ | 3524 | 1:20 |
| | (HP: 1) | (89.04)²⁺ | 0 | (152.50)²⁺ | 0 | (201.65)²⁺ | 0 | |

The effect of C₈-QAT labeling on electrospray ionization of tryptic peptides. The impact of C₈-QAT labeling on electrospray ionization of typical tryptic peptides was determined. This was achieved using native and C₈-QAT labeled transferrin digests with an equimolar distribution of peptides at a concentration of 10⁻⁶ M. The labeled and un-labeled digests were mixed in a 1 to 1 ratio and separated on the C₁₈ Zorbax column before electrospray ionization and introduction into the mass spectrometer. MASCOT was used for peptide identification after including as variable modifications new masses for the tagged amine groups at the N-terminus and on lysine. Peptides identified by this procedure are listed in Table 5. Rᵢ values were calculated and used to determined differences in ionization efficiency.

The average increase in ionization was estimated to be 70-fold. A 500-fold increase in ionization efficiency was observed for the peptide ADRDQYELLCLDNTRKPVDEYK. This peptide was labeled on three different amino acids, i.e., the N-terminus and both lysine residues. However, the enhancement of ionization seen with this peptide was probably not due to being derivatized at multiple sites. Ionization of the peptide DCHLAQVPSHTVVAR with a single tag was enhanced 453-fold. The number of sites tagged seemed not to correlate with increases in ionization efficiency throughout this work. For example, the peptide CLKDGAGDVAFVK is triply tagged (at N-terminus and both lysine residues) but showed only a 2-fold increase in ionization efficiency. Nonetheless, there was an overall increase in ionization efficiency.

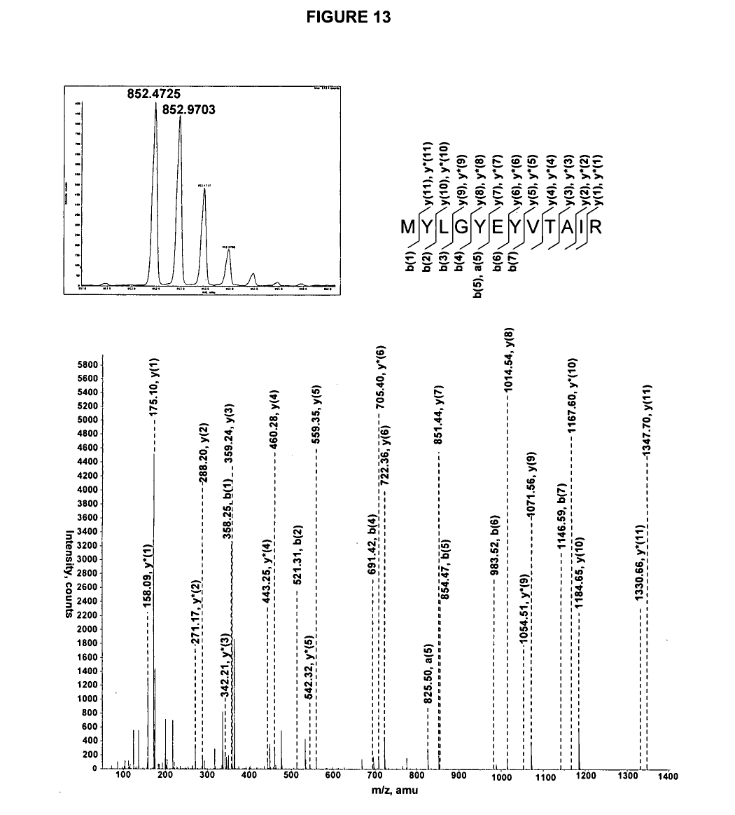

MS/MS analysis of C₈-QAT labeled peptides from transferrin. The effect of C₈-QAT labeling on CID induced fragmentation was examined using peptides from the transferrin digest. The MS/MS spectrum and the peak map for MYLGYEYVTAIR is shown in FIG. 13, showing that all the y ions and 7 b ions were found. FIG. 13 is a graph depicting the MS/MS spectrum for the peptide MYLGYEYVTAIR. All the y ions and 7 b ions were found for this peptide and mapped on the spectrum. The mass of methionine labeled with C₈-QAT reagent was calculated using both y and b ions.

The mass of C₈-QAT labeled methionine at the N-terminus of the peptide can be calculated using either the b(1) ion or the difference between the precursor ion mass and the mass of y(11) fragment ion→(852.45)²⁺−1347.70 y(11)−1.0079. A proton is subtracted from the mass of y(11) ion because this fragment has absorbed a proton to be positively charged following loss of the quaternary amine tag. All the peptides in Table 5 were identified from their MS/MS spectra. A total of 25 peptides were identified using manual sequencing.

Table 5 is a list of native and C₈-QAT labeled transferrin peptides identified by LC/MS/MS analysis. The average increases in ionization efficiency of these peptides were calculated to be 70-fold. The areas under the peak for all charge states of each peptide were summed and used as a total ionization measure. The ratios were calculated by dividing the total peak areas for each peptide by the total peak area of the native peptide. *AA: C₈QAT labeled N-terminal; E*: sodiated glutamic acid; D*: sodiated aspartic acid; M*: oxidized methionine to methionine sulfoxide; K*: C₈QAT labeled; AA°: sodiated C-terminal.

TABLE 5
Native and C₈-QAT labeled transferrin peptides identified by LC/MS/MS analysis

| | | | | | | | Rᵢ based |
|---|---|---|---|---|---|---|---|
| | Native | | | | | | ratio Native: |
| # | peptide | M/Z | RT | Labeled peptide | M/Z | RT | C₈-QAT |
| 1 | AD*RD*QYE* | (917.44)³⁺ | 13 | A*DRD*QYE*LLCLD | (852.48)⁴⁺ | 52 | 1:500 |
| | LLCLDNTRK | | | NTRK*PVDEYK* | | | |
| | PVDEYK | | | | | | |
| 2 | CD*E*WSVN | (633.87)²⁺ | 40 | *CD*E*WSVNSVGK* | (860.47)²⁺ | 45 | 1:72 |
| | SVGK | | | | | | |
| 3 | CLKD*GAGD | (683.82)²⁺ | 18 | *CLK*DGAGDVAFVK* | (1001.56)²⁺ | 54 | 1:2 |
| | *VAFVK | | | | | | |
| 4 | CQSFRDHM*K | (584.27)²⁺ | 22 | *CQSFRD*HM*K* | (821.34)²⁺ | 44 | 1:8 |
| 5 | CQSFRD*HMK | (1173.96)²⁺ | 18 | *CQSFRD*HMK* | (813.46)²⁺ | 38 | 1:43 |
| 6 | DCHLAQVPS | (817.01)²⁺ | 30 | *DCHLAQVPSHTVV | (929.97)2* | 49 | 1:453 |
| | HTVVAR | | | AR | | | |
| 7 | D*CHLAQVP | (839.04)²⁺ | 40 | *D*CHLAQVPSHTVV | (951.61)²⁺ | 54 | 1:19 |
| | SHTVVAR* | (559.67)³⁺ | | AR° | (634.75)³⁺ | | |
| 8 | D*SGFQM*N | (710.01)²⁺ | 27 | *D*SGFQM*NQLRGK* | (936.06)²⁺ | 55 | 1:21 |
| | QLRGK | | | | | | |
| 9 | EDLIWELLN | (1035.52)²⁺ | 49 | *EDLIWELLNQAQEH | (1261.84)²⁺ | 70 | 1:1.6 |
| | QAQEHFGK | | | FGK* | (841.56)³⁺ | | |
| 10 | E*D*PQTFYY | (901.43)²⁺ | 20 | *ED*PQTFYYAVAVV | (820.04)³⁺ | 27 | 1:29 |
| | AVAVVKK | | | K*K* | (1229.56)²⁺ | | |
| 11 | HSTIFENLANK | (637.32)²⁺ | 20 | *HSTIFENLANK* | (863.43)²⁺ | 51 | 1:14 |
| 12 | SASDLTWDNLK | (625.31)²⁺ | 27 | *SASDLTWDNLK* | (851.38)²⁺ | 57 | 1:79 |
| 13 | SASDLTWD* | (636.43)²⁺ | 43 | *SASD*LTWDNL | (862.54)²⁺ | 50 | 1:37 |
| | NLK | | | K* | | | |
| 14 | TVRWCAVS | (539.68)³⁺ | 37 | *TVRWCAVSE*HE*A | (1057.02)²⁺ | 57 | 1:2.2 |
| | EHEATK | 1008 | | TK* | | | |
| 15 | VPPRMD*AK | (468.25)²⁺ | 29 | *VPPRMD*AK* | (694.41)²⁺ | 47 | 1:8 |
| 16 | RLAVGALLV | (858.41)³⁺ | 47 | *RLAVGALLVCAVLG | (1008.92)³⁺ | 69 | 1:8 |
| | CAVLGLCLA | | | LCLAVPD*K*TVR* | | | |
| | VPD*KTVR | | | | | | |
| 17 | AIAANEADA | (989.28)⁴⁺ | 46 | *AIAANEADAVTLDA | (1158.95)⁴⁺ | 70 | 1:3 |
| | VTLDAGLVY | | | GLVYDAYLAPNNLK* | | | |
| | DAYLAPNNL | | | PVVAEFYGSK* | | | |
| | KPVVAEFYG | | | | | | |
| | SK | | | | | | |
| 18 | KCSTSSLLE* | (921.94)²⁺ | 24 | *K*CSTSSLLE*ACTF | (765.76)³⁺ | 61 | 1:20 |
| | ACTFRRP | | | RRP° | (1148.13)²⁺ | | |
| 19 | WCAVSEHE* | (952.50)²⁺ | 38 | *WCAVSEHE*ATK*C | (786.07)³⁺ | 48 | 1:14 |
| | ATKCQSFR | | | QSFR | (1178.58)²⁺ | | |

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of biochemistry, are within the scope of this invention. All publications, patents, and patent applications cited herein are incorporated by reference in their entirety for all purposes.

What is claimed is:

1. A reagent comprising:
an N-hydroxysuccinimide ester of N-alkyl-nicotinic acid (Cₙ-NA-NHS).

2. The reagent of claim 1, wherein the alkyl chain of the N-hydroxysuccinimide ester of N-alkyl-nicotinic acid comprises 1 to 4 carbon atoms.

3. The reagent of claim 1 or 2, comprising at least one deuterium atom.

4. A method of analyzing an amino acid, comprising:
a) contacting the amino acid with an N-hydroxysuccinimide ester of N-alkyl-nicotinic acid (Cₙ-NA-NHS) to derivatize the amino acid, and
b) detecting the resultant derivative by a light absorbance method.

5. The method of claim 4, further comprising the step of c) purifying the resultant derivative with reversed phase chromatography.

6. The method of claim 5, further comprising the step of d) analyzing the resultant derivative with electrospray ionization mass spectrometry.

7. The method of any of claims 4-6, wherein the light absorbance method comprises UV or fluorescence methods.

8. The method of any of claims 4-7, wherein the alkyl chain of the N-hydroxysuccinimide ester of N-alkyl-nicotinic acid comprises 1 to 4 carbon atoms.

9. A method for affixing a tag to an amino acid, comprising:
contacting the amino acid with a derivatization reagent comprising an N-hydroxysuccinimide ester of N-alkyl-nicotinic acid (Cₙ-NA-NHS).

10. The method of claim 9, wherein the alkyl chain of the N-hydroxysuccinimide ester of N-alkyl-nicotinic acid comprises 1 to 4 carbon atoms.

11. A reagent comprising:
a quaternary amine;
an n-octyl chain bonded to the quaternary amine; and
a peptide binding group, the reagent covalently binding to a peptide through the peptide binding group.

12. The reagent of claim 11 comprising a [3-(2,5)-dioxopyrrolidin-1-yloxycarbonyl)-propyl]-dimethyloctylammonium (C₈-QAT).

13. A method of analyzing a peptide, comprising:
a) contacting the peptide with a reagent of claim 11 to derivatize the peptide, and
b) detecting the resultant derivative by a light absorbance method.

14. The method of claim 13, wherein the reagent is [3-(2,5)-dioxopyrrolidin-1-yloxycarbonyl)-propyl]-dimethyloctylam monium (C₈-QAT).

15. The method of claim 13, wherein the reagent is activated with N-hydroxysuccinimide (NHS) prior to contacting the peptide.

16. The method of claim 13 or 14, further comprising the step of c) analyzing the resultant derivative with reversed phase chromatography.

17. The method of any of claims 13-16, further comprising the step of d) analyzing the resultant derivative with electrospray ionization mass spectrometry.

18. The method of any of claims 13-17, wherein the light absorbance method comprises UV or fluorescence methods.

19. A method for affixing a tag to a peptide, comprising:
contacting the peptide with a reagent comprising a quaternary amine with an n-octyl chain bonded to the quaternary amine, the reagent having a peptide binding group, the reagent covalently binding to the peptide through the peptide binding group.

20. The method of claim 19, wherein the reagent is [3-(2,5)-dioxopyrrolidin-1-yloxycarbonyl)-propyl]-dimethyloctylammonium (C₈-QAT).
